(19) United States
(12) Patent Application Publication
(10) Pub. No.: US 2014/0308726 A1
(43) Pub. Date: Oct. 16, 2014

(54) Controlling Bacterial Biofilms in Ethanol Production

(71) Applicant: ANITOX CORPORATION, LAWRENCEVILLE, GA (US)

(72) Inventor: Julio Pimentel, Buford, GA (US)

(21) Appl. No.: 14/356,109

(22) PCT Filed: Oct. 29, 2012
(86) PCT No.: PCT/US2012/062481

§ 371 Date: May 2, 2014

Related U.S. Application Data
(60) Provisional application No. 61/558,047, filed on Nov. 10, 2011.

(51) Int. Cl. C12P 7/10; C12P 7/06

(52) U.S. Cl. CPC C12P 7/10; C12P 7/06
USPC 435/165; 435/161; 435/255.1

(57) ABSTRACT

A high yield method for fermenting carbohydrate to ethanol and prevention and/or disruption of biofilms, comprising: a) mixing a fermentation feedstock with a fermentation broth containing yeast and/or an enzyme, b) treating said mixture by adding a composition to the fermentor containing: 10-90 wt. % of an aldehyde selected from the group consisting of formaldehyde, para-formaldehyde, glutaraldehyde, another antimicrobial aldehyde, and mixtures thereof, 1-50 wt. % of a surfactant having an HLB from 4 to 18, 0-20 wt. % of an antimicrobial terpene, or essential oils, 1-50 wt. % of organic acids selected from Ci to C24 fatty acids, their salts, glycerides and esters thereof, and 1-50 wt. % water; wherein the concentration of aldehyde in the fermentor is from about 0.25 to 3 kg/MT of fermentation feedstock, and c) isolating ethanol.

### BACKGROUND OF THE INVENTION

1. Field of the Invention

An improved method for producing ethanol, by treating carbohydrate material, carbohydrate broth or carbohydrate slurry throughout the fermentation process with a composition containing an aldehyde, a fatty acid, a terpene and a surfactant. Ethanol yields are improved by controlling the formation of biofilms and destroying pre-existing biofilms in the fermentation system.

2. Background

High oil prices have brought about an increase on the search for renewable fuels. Ethanol is one of these renewable fuels which, when mixed with gasoline, can decreased the need for imported oil.

In 2009, the Renewable Fuels Standard (RFS) called for blending 11.1 billion gallons of ethanol and other biofuels into the U.S. motor fuels market to satisfy future demand. This will increase the level of corn needed by the industry, and will require plant capacity to be increased as well. In 2010, the USA's annual operating capacity increased by 2.7 billion gallons, a 34% increase over 2007 levels.

Ethanol, a promising biofuel from renewable resources, is produced from the starch of cereal grains (corn, sorghum, wheat, triticale, rye, malted barley, rice), tuber crops (potatoes) or by direct use of the sugar in molasses, sugar cane juice or sugar beet juice. Ethanol can also be produced by fermentation of cellulose-based material (switchgrass, pine trees). Ethanol from grasses or bagasse is now commercially available by the use of high temperature de-lignification of plant materials and the use of enzymes and special yeast that can use C-5 sugar and convert it to C-6 sugar or to ethanol. The use of wood i.e. pine trees, is still in its infancy because of the high cost of converting hardwood into easy-to-use material.

Eighty percent of the world's ethanol is produced by Brazil and the USA. Of this, 60% is produced by yeast fermentation of corn or sugar cane juice. Ethanol production through anaerobic fermentation of a carbon source by the yeast Saccharomyces cerevisiae is one of the best known biotechnological processes and accounts for more than 35 billion liters of ethanol per year worldwide (Bayrock, 2007).

Ethanol production from cereal grains begins with the hydrolysis of starch resulting in the conversion of amylose, a mostly linear α-D-(1-4)-glucan, and branched amylopectin, a α-D-(1-4)-glucan which has α-D-(1-6) linkages at the branch point, into fermentable sugars which are subsequently converted to ethanol by yeast (Majovic, 2006) or bacteria (Dien, 2003). Bacteria can convert cellulose-containing material into fermentable sugars for the production of ethanol; these include Zymomonas spp., genetically engineered E. coli, Klebsiella oxytoca, Zymomonas mobilis, Acetivibrio celluloyticus and others (Dien, 2003). Ethanol from sugarcane does not require the use of enzymes since yeast easily converts sucrose to ethanol and CO₂

Dry milling and wet milling are the two primary processes used to make ethanol from cereal grains in the United States. In the dry milling process, the entire corn (Zea mays) kernel or other starchy material is ground into flour and mixed with water to form a slurry. The mixture is then steam cooked to gelatinize the starch and decrease bacterial contamination. This mixture is then cooled and transferred to fermenters where yeast and enzymes are added to convert the sugars to ethanol. After fermentation, the resulting mixture is transferred to distillation columns where the ethanol is separated. The solids remaining after fermentation and ethanol separation are processed into distiller's dried grains with solubles (DDGS), which is used for animal production, e.g. poultry, swine, and cattle feed. More than 80% of today's ethanol capacity uses the dry mill process (RFS, 2006).

In wet milling the grain is soaked or steeped in water to facilitate separation of the grain into its basic nutritional components, such as corn germ, fiber, gluten and starch. After steeping, the corn slurry is processed through a series of grinders and the components are separated. The gluten component is filtered and dried to produce corn gluten meal (CGM), a high-protein product used as a feed ingredient in animal operations. The starch and any remaining water from the corn slurry are then processed in one of three ways: Fermented into ethanol, dried and sold as dried or modified corn starch, or processed into corn syrup (RFS, 2006). Both the wet and dry mill processes use only the starch portion of the corn kernel for ethanol production. The remaining protein, fat, fiber and other nutritional components remain available for use as animal feed.

A process called raw starch hydrolysis (dry grinding) converts starch to sugar which is then fermented to ethanol, bypassing conventional starch gelatinization conditions. The enzymes used in the saccharification/fermentation are fungal alpha amylase and glucoamylase (amyloglucosidase) (Thomas, 2001). This simultaneous saccharification and fermentation allows for higher concentrations of starch to be fermented and results in higher levels of ethanol (Maye, 2006).

Sugar cane, “saccharuk officinarum”, is the cheapest raw material for renewable energy production. Comparing sugar cane and corn, the sugar cane can yield 5000-7000 liters/Ha/year of ethanol while corn's ethanol yield is 3000 liters/Ha/year (Lee and Bressa, 2006). Brazil and India are the main producers of ethanol from sugar cane. The production process begins with cultivating and harvesting sugarcane at a cane field. The cane is then processed at a sugar/ethanol mill, where cane stalks are washed with acidified water, then shredded and crushed to extract the cane juice. The bagasse, which is the resulting cane after the juice has been extracted, can be used to produce steam and generate electricity within the plant or sold to utility grids. In other mills, the cellulose from bagasse can be used to produce ethanol. After sugarcane juice is extracted it is transformed into alcohol through a fermentation process using yeasts as the catalyst. Sugar from sugarcane is readily available to yeast so fermentation requires only between 4 to 12 hours, compared to 72 hours for fermentation using cereal grains. Fermentation can be conducted in batches or continuously, using open or closed fermentation tanks. After fermentation, the sugarcane ethanol is distilled from other byproducts resulting in a level of purity of approximately 95%.

Another source for ethanol production is the sugar beet ,“beta vulgaris.” Sugar beet can be stored for one to three days, depending on the temperature and the method of storage, whereas sugar cane must be processed immediately after harvesting due to sugar losses. During the production of sugar from beet, slicing of the beet can cause some sugar to undergo breakdown to inverted sugar and then into acids, reducing sugar yields. In order to decrease bacterial action, it is known to use formaldehyde (50 to 100 ppm) and a pH adjustment. This method is used only during sugar production, not in a combined process of sugar and ethanol production. Arvanitis et al. (2004) suggests the use of formaldehyde or other cost effective disinfectant for the control of dextran produced by bacteria. Dextran inhibits crystallization of sugar. It also suggests controlling bacteria to preserve the sugar level if sugar beets are stored for long time. However all experimental data was from 7-day studies. Storage of sugar beets caused sugar levels to decrease due to bacterial contamination and dextran production. The reference teaches the use of 3.7% formaldehyde to store sugar beet longer to prevent bacterial contamination in sugar produced from sugar beet. There is no suggestion of using more concentrated formaldehyde (Arvantis et al. used 3.7% instead of 37%) for the production of ethanol from sugar beet. The MIC using formaldehyde was from 25-500 mg/lt. If the working solution is 3.7%, then the amount of formaldehyde added is only 0.925 mgr (25 mg/lt*0.037) to 18.5 mgr (500 mg/lt*0.037).

A variety of gram positive and gram negative bacteria have been isolated from fuel ethanol fermentation including species of Lactobacillus, Pediococcus, Staphylococcus, Enterococcus, Acetobacter, Gluconobacter and Clostridium (Bischoff, 2009). Almost two thirds of the bacteria isolated were species of lactic acid bacteria, e.g. Lactobacillus (Skinner, 2007). In sugar cane, Leuconostoc has been reported to negatively influence ethanol yield. The contamination of carbohydrate slurry during the course of alcoholic fermentation results in a) decreased ethanol yield, b) increased channeling of carbohydrates for the production of glycerol and lactic acids, c) a rapid loss of the yeast viability after exhaustion of fermentable sugars, and d) decreased proliferation of yeast in the corn slurry in which the contaminating Lactobacilli spp. have already grown to a high number (Thomas, 2001).

In a survey conducted by Skinner and Leathers (2004), 44-60% of the contaminants in the wet mill process were identified as Lactobacilli spp. In the dry mill process, 37 to 87% of the contaminants were identified as Lactobacilli spp. Another survey of bacterial contaminants of corn-based plants in the US found that bacterial loads in a wet mill facility were approximately 10⁶ cfu/ml corn slurry while those at dry-grind facilities could reach 10⁸ cfu/ml corn slurry (Bischoff, 2007; Chang, 1997).

Lactobacilli spp. contamination in the range of 10⁶ to 10⁷ cfu/mlml corn slurry can reduce ethanol yield by 1-3%. In industry, even with an active bacterial control program to control the proliferation of Lactobacilli spp., carbohydrate losses to Lactobacilli spp. can make the difference between profitability and non-profitability (Bayrock, 2007). Lactobacilli spp. not only tolerate low pH, high acidity and relatively high concentrations of ethanol, but they also multiply under conditions of alcoholic fermentation (Thomas, 2001). Bacterial contaminants compete for growth factors needed by yeast and also produce by-products that are inhibitory to yeast, particularly lactic and acetic acids.

The presence of Lactobacillus byproducts, i.e. acetic and lactic acids, during fermentation affects yeast growth and metabolism, and it has been suggested as one of the causes of stuck or sluggish fermentation (Thomas, 2001). If the lactic acid content of the corn slurry approaches 0.8% and/or acetic acid concentration exceeds 0.05%, the ethanol producing yeast are stressed (Bayrock, 2007). Lactobacilli spp. may stress yeast cells, which release nutrients, particularly amino acids and peptides that can stimulate bacterial growth (Oliva-Neto, 2004). A lactic acid concentration of 8 g/L in a beet molasses batch fermentation reduced yeast viability by 95% and alcohol production rate by 80% (Bayrock, 2001).

The presence of Lactobacillus in the ethanol fermentation can decrease ethanol yield by 44% after 4 days of pH controlled operation. This coincides with an increase in L. paracasei to >10¹⁰ cfu/ml and a fourfold increase in lactic acid concentration to 20 g/L. An 80% reduction in yeast density was seen with concentrations of ethanol, lactic acid and acetic acid of 70, 38 and 7.5 g/L respectively (Bayrock, 2001).

De Oliva-Neto and Yokoya (1994) evaluated the effect of bacterial contamination on a batch-fed alcoholic fermentation process. They showed that L. fermentum will strongly inhibit commercial baker's yeast in a batch-fed process. When the total acid (lactic and acetic) exceeded 4.8 g/L it interfered with yeast bud formation and viability with 6 g/L decrease in alcoholic efficiency.

Others have shown that: a) a 10⁶ Lactobacilli spp./ml corn slurry results in approx 1% v/v reduction in the final ethanol produced by yeast (Narendranath, 2004), b) challenging the fermentation system with 10⁸ cfu L. fermentum/ml in the corn slurry decreased ethanol yield by 27% and increased residual glucose from 6.2 to 45.5 g/L (Bischoff, 2009), and c) the use of 10⁵ cfu Lactobacilli spp./ml produced an 8% reduction in ethanol yield and a 3.2 fold increase in residual glucose (Bischoff, 2009).

Sugar cane depending on harvesting, storage and environmental conditions can suffer from Leuconostoc deterioration which resulted in a decrease in ethanol yield and increase formation of dextran (glucose polysaccharide) that inhibit crystallization of sugar. Leuconostoc is also present on sugar beet process (Eggleston et. al. 2008).

Conditions in the fermentation/liquidfication tanks are optimum for bacterial growth. Contamination generally originates from harvesting of the carbohydrate material. Washing the material may help lower the contamination level (Mayes, 2006). Other methods to control bacteria include the addition of more yeast culture, stringent cleaning and sanitation, acid washing of yeast destined for reuse, and the use of antibiotics during fermentation (Hynes, 1997). An increased yeast inoculation rate of 3×10⁷ cfu/ml corn slurry resulted in greater than 80% decrease in lactic acid production by L. plantarum and greater than 55% decrease in lactic acid production by L. paracasei, when corn slurry was infected with 1×10⁸ Lactobacilli spp./ml (Narendranath, 2004; Bischoff, 2009).

Currently, virginiamycin is the only approved antibiotic known to be used at the dry-grind plant (Bischoff, 2007). The recommended dose of virginiamycin in fuel ethanol fermentations is generally 0.25 to 2.0 ppm (Bischoff, 2009) but the Minimum Inhibitory Concentration (MIC) varies from 0.5 to greater than 64 ppm (Hynes, 1997).

Various agents have been tested for control of bacterial contaminants in laboratory conditions including antiseptics such as hydrogen peroxide, potassium metabisulfite, and 3,4,4′-trichlorocarbanilide and antibiotics such as penicillin, tetracycline, monensin and virginiamycin. Penicillin and virginiamycin are commercially sold today to treat bacterial infections of fuel ethanol fermentation and some facilities use these antibiotics prophylactically (Skinner, 2004).

If no antibiotics are used, a 1% to 5% loss in ethanol yield is common. A fifty million-gallon fuel ethanol plant operating with a lactic acid level of 0.3% w/w in its distiller's beer is losing approximately 570,000 gallons of ethanol every year due to bacterial contamination (Maye, 2006). In the absence of an antibiotic, bacterial numbers increased from 1×10⁶ cfu/ml to 6×10⁶cfu/ml during a 48 hour fermentation period and 5.8 mg lactic acid was produced (Hynes, 1997).

A bacterial control program involves the use of virginiamycin. Some characteristics of virginiamycin are: a) it is effective against a number of microorganisms including Lactobacilli spp. at low concentrations, e.g., 0.3 to 5 ppm, b) the microorganisms do not tend to develop resistance, c) it does not significantly inhibit the yeast, d) it is not affected by the pH or alcohol concentration, and e) it is inactivated during ethanol distillation, therefore no residue remains in the alcohol or distilled grains (Bayrock, 2007; Narendranath, 2000; Hynes, 1997). Decreased susceptibility to virginiamycin has been observed in Lactobacilli spp. isolated from dry-grind ethanol plants that use virginiamycin, and the emergence of isolates with multi-drug resistance to both penicillin and virginiamycin has also been reported (Bischoff 2009).

L. fermentum could be selectively controlled by hydrogen peroxide at concentrations of 1 to 10 mM in an ethanol fermentation process (Narendranath, 2000). Lactobacillus does not have the enzyme catalase, so it cannot decompose hydrogen peroxide and therefore is unable to eliminate its toxic effect (Narendranath, 2000).

Urea hydrogen peroxide (UHP) has been used as an antiseptic for topical applications on wounds and against gingivitis and dental plaque (Narendranath, 2000) and also serves as an antibacterial during fermentation. UHP not only exhibits excellent bactericidal activity against Lactobacillus but also has an important advantage of providing usable nitrogen in the form of urea for stimulating yeast growth and fermentation rates (Narendranath, 2000).

Other methods of controlling bacterial contamination include the use of sulfites. Sulfites demonstrate bactericidal activity only in the presence of oxygen and were more effective in killing facultative L. casei which possess high levels of hydrogen peroxide related enzymes, including peroxidase (Chang, 1997). Bacterial load was also decreased when the concentration of sulfite ranged from 100 to 400 mg/L but only in the presence of oxygen. This concentration did not affect yeast populations (Chang, 1997).

An agent present in the supernatant of yeast cultures reduces the growth of Lactobacilli spp. This compound has not yet been characterized, though it is known to be resistant to freezing, unstable at high temperatures and destroyed when held at 90° C. for 20 minutes (Oliva-Neto 2004).

Succinic acid by itself at levels of 600 mg/L reduces Lactobacillus concentrations by 78%, in the presence of ethanol that reduction is up to 96% (Oliva-Neto 2004).

A microbial adherence inhibitor in the form of fowl egg antibodies and specific to lactic acid-producing microorganisms has been developed for use in fermenters (Nash 2009).

Laboratory studies have shown that antibodies, sulfite and peroxide products can be beneficial in controlling Lactobacilli spp., a problem with these products is the decrease in concentration due to oxidation and decomposition of the chemicals which will require constant monitoring of the whole process of fermentation in order to maintain an effective concentration.

U.S. Pat. No. 7,955,826 suggests the use of a monoterpene and a surfactant to improve production of ethanol. The monoterpene is d-limonene. The composition is added to the fermentation medium resulting in reduced cleaning requirements. The composition is a water/oil emulsion added to a level of 0.1-1000 ppm. It is also suggested to improve the viability of yeast and is added to corn fermentation media, the emulsion containing 1-70% d-limonene, 0.2-25% surfactant and the balance water.

To prevent sugar cane deterioration a combination of 8.6 ppm Nisin and 0.1% Tween 20 can be used to delay the lag phase of lactobacillus for 12 hours (Franchi et. al., 2006). The use of 10 ppm Kamoran (tade name of monensin) or a mixture of penicillin 10 ppm and tetracycline have been used to prevent sugar cane deterioration (Payot, 2004). In a related study, out of five commercially available antimicrobial products, only two containing formaldehyde (3.7%) or a quaternary ammonium-isopropanol (3.5%), showed similar effectiveness against lactic bacteria in sugar cane facilities (Arvanitis, et. al., 2004).

In a dry-grind fuel ethanol plant that uses virginiamycin, six strains of Lactobacillus fermentum, two strains of L. johnsonii and one strain of L. mucosae and L. amylovorus were found all around the fermentation system. It was suggested that biofilms may play a role in the persistence of contaminants in ethanol production facilities (Rich et. al. 2011).

Despite efforts to prevent contamination through cleaning and disinfecting saccharification tanks and continuous yeast propagation systems, biofilms can act as reservoirs of bacteria that continuously reintroduce contaminants (Bischoff, 2009). Biofilms can occur in many locations; in the human body, for example, they occur in gums, teeth, and ears and can be responsible for infections in that area. Biofilm cells are organized into structured communities enclosed in a matrix of extracellular material. They are phenotypically different from planktonic or suspended cells. They resist host defenses and display decreased susceptibility to antimicrobial agents (Berit et. al. 2002). Damaged lines or pipes that are abraded or scratched create surfaces where organisms can more easily attach. Biofilms are the source of much of the free-floating bacteria in drinking water and machinery, especially in pipes. Once bacteria colonize, they start forming a glycocalyx matrix that holds water, making a film of gelatinous and slippery consistency. This gel-like film encloses the microbial cell and may act as a barrier against the penetration of sanitizers and antimicrobials (Perez-Conesa, et.al. 2006). A review of microbial biofilms can be found in Davey and O'Toole (2000).

Several US patents describe products to control biofilms. U.S. Pat. No. 6,830,745 teaches using a couple of enzymes systems, one which disrupts biofilm structure and another having a bactericidal effect. U.S. Pat. No. 8,012,461 teaches a biofilm remover which is an aqueous solution containing a quaternary halide surfactant and a source of bromide ions. U.S. Pat. No. 7,165,561 discloses an enzyme and surfactant to decrease and inhibit the growth of biofilms in crossflow filtration systems. US Published Application No. 2011/0123462 discloses the use of unsaturated long chain alcohols and/or aldehydes for the disruption of biofilms, the solutions containing 0.005% to 5% of the active ingredient, preferably 0.05% and 22% ethanol and 77% water.

Controlling the formation of biofilms is important to do throughout the fermentation system, from cutting the sugar cane or sugar beet all the way through the final product. The present invention can be used during all of these steps of ethanol fermentation. In the case of sugar cane, it can be added to the first juice obtained after cutting and pressing the cane. It can be used during the transferring of juice to the cooling area. It can be used when mixing the juice to obtain the right sugar concentration before going to the fermentation vessel. It can be used while filling up the fermentation vessel with juice or in combination with the yeast broth. Other points of addition for the present invention can be used with the same results, i.e. improved ethanol yield by controlling biofilms. The present invention can prevent the formation of biofilms as well as disrupt established biofilms.

### REFERENCES

Arvanitis, N., C. Z. Kotzamanidis, G. N. Skaracis and A. D. Karagouni. The effectiveness of commercial antimicrobial compounds against saccharolytic microorganisms isolated from beet sugar production line. World J. Microbiology & Biotechnology 2004, 20: 291-296.

Bayrock, D., 2007. Method of reducing the growth of lactobacillus in a process of ethanol production by yeast fermentation comprising adding a pristinamycin type antimicrobial agent and/or a polyether ionophore antimicrobial agent dissolved in an organic solvent. PCT patent # WO 2007/145858

Bayrock, D. P., K. C. Thomas and W. M. Ingledew. Control of Lactobacillus contaminants in continuous fuel ethanol fermentations by constant or pulsed addition of penicillin. G. App. Microbiol. Biotechnol 2003, 62: 498-502.

Bayrock, D. and W. M. Ingledew. Changes in steady state on introduction of a lactobacillus contaminant to a continuous culture ethanol fermentation. J. Industrial Microbiology and Biotechnology 2001, 27: 39-45.

Berit, A. G. S. Baillie and L. J. Douglas. Mixed species biofilms of Candida albicans and Staphylococcus epidermis. J. Med Microbiol 2002, 51: 344-349.

Bischoff, K. M., S. Liu, T. D. Leathers and R. E. Worthington. Modeling bacterial Contamination of Fuel Ethanol Fermentation. Biotechno. Bioeng. 2009, 103: 117-122.

Bischoff, K. M., K. A. Skinner-Nemec and T. D. Leathers. Antimicrobial susceptibility of Lactobacillus species isolated from commercial ethanol plants. J. Ind. Microbiol. Biotechnol. 2007

Chang I. N., B. H. Kim and P. K. Shin. Use of sulfite and hydrogen peroxide to control bacterial contamination in ethanol fermentation. Applied and Environmental Microbiology 1997, 63(1): 1-6.

Davey, W. E. and G. A. O'Toole. Microbiology and Molecular Biology Reviews 2000, 64(4): 847-867.

Dien, B. S., M. A. Cotta and T. W. Jeffries. Bacteria engineered for fuel ethanol production: current status. Appl. Microbiol. Biotechnol. 2003, 63: 258-266.

Eggleston, G., M., P. G. Moerl Du Boil and S. N. Waldford. A review of sugar cane deterioration in the United States and South Africa. Proc. S. Afr. Sug. Technol. Ass. 2008, 81: 72-85.

Franchi, M. A., G. E. Serra and M. Cristianini. The use of biopreservatives in the control of bacterial contaminants of sugarcane alcohol fermentation. 2006, 68(7):2310-2315.

Hynes, S. H., Kjarsgaard, K. C. Thomas and W. M. Ingledew. Use of virginiamycin to control the growth of lactic acid bacteria during alcohol fermentation. J Industrial Microbiology and Biotechnology 1997, 18: 284-291.

Lee T. S. G. and E. A. Bressan. Sugar Tech 2006, 8(4): 195-196.

Majovic, L, S. Nikolic, M. Rakin and M. Vukasinovic. Production of Bioethanol from Corn Meal Hydrolyzates. Fuel 2006, 85: 1750-1755.

Maye, John P., 2006. Use of hop acids in fuel ethanol production. US patent application #20060263484

Narendranath, N. V. and R. Power. Effect of yeast inoculation rate on the metabolism of contaminant Lactobacilli spp. during fermentation of corn corn slurry. J. Ind. Microbiol. Biotechnol. 2004, 31: 581-584.

Narendranath, N. V., K. C. Thomas and W. M. Ingledew. Urea hydrogen peroxide reduces the number of Lactobacilli spp., nourish yeast, and leaves no residues in the ethanol fermentation. Applied and Environmental Microbiology 2000, 66(10): 4187-4192.

Nash, P. 2009. Immunogen adherence inhibitor directed to lactobacillus organisms and method of making and using it. United States Patent Application #20090117129

Oliva-Neto, P., M. A. Ferreira and F. Yokoya. Screening for yeast with antibacterial properties from ethanol distillery. Bioresource Technology 2004, 92: 1-6.

Payot, T. 2004. Kamoran using in sugar beet production to improve the quality of diffusion step UNGDA, www.ungda.com.

Perez-Conesa, D., L Mclansboough and J. Weiss. Inhibition and inactivation of Listeria monocytogenes and Escherichia coli O157:H7 colony biofilms by micellar-encapsulated eugenol and carvacrol. J. Food Protection 2006, 69(12): 2947-2954.

Rich, J. O., T. D. Leathers, M. S. Nunnally and K. M. Bischoff. Rapid evaluation of the antibiotic susceptibility of fuel ethanol contaminant biofilms. Bioresource Technology 2011, 102: 1124-1130.

RFA “Renewable Fuels Association 2006 and 2009.

Skinner-Nemec, K. A., N. N Nichols and T. D. Leathers. Biofilm formation by bacterial contaminants of fuel ethanol production. Biotechnol. Lett. 2007, 29: 379-383.

Skinner, K. A. and T. D. Leathers. Bacterial Contaminants of Fuel Ethanol Production. J. Ind. Microbiol. Biotech. 2004, 31: 401-408.

Thomas, K. C., S. H. Hynes and W. M. Ingledew. Effect of Lactobacilli spp. on yeast growth, viability and batch and semi-continuous alcoholic fermentation on corn corn slurry. J. Applied Microbiology 2001, 90: 819-828.

### SUMMARY OF THE INVENTION

An object of the invention is to provide a chemical composition that prevents and/or disrupts biofilm formation during ethanol production, by reducing or not allowing establishment of bacteria on solid surfaces.

Another object is to A high yield method of fermenting carbohydrate to ethanol in a fermentor, comprising:

a) mixing a fermentation feedstock with a fermentation broth containing yeast and/or an enzyme,

b) treating said mixture by adding a composition to the fermentor containing:
10-90 wt. % of an aldehyde selected from the group consisting of formaldehyde, para-formaldehyde, glutaraldehyde, another antimicrobial aldehyde, and mixtures thereof,
1-50 wt. % of a surfactant having an HLB from 4 to 18,
0-20 wt. % of an antimicrobial terpene, or essential oils,
1-50 wt. % of organic acids selected from C₁ to C₂₄ fatty acids, their salts, glycerides and esters thereof, and
1-50 wt. % water;
   wherein the concentration of aldehyde in the fermentor is from about 0.25 to 3 kg/MT of fermentation feedstock, and

c) isolating ethanol.

Another object is to provide a fermentation broth or slurry, comprising:

a) carbohydrate feedstock to be fermented, yeast, and/or an enzyme, and

b) a treatment composition containing:
10-90 wt. % of an aldehyde selected from the group consisting of formaldehyde, para-formaldehyde, glutaraldehyde, another antimicrobial aldehyde and mixtures thereof,
1-50 wt. % of a surfactant having an HLB from 4 to 18,
1-20 wt. % of an antimicrobial terpene, or essential oils,
1-50 wt. % of organic acids selected from C₁ to C₂₄ fatty acids, their salts, glycerides and esters thereof, and
1-50 wt. % water;
   wherein the concentration of aldehyde is from about 0.25 to 3 kg/MT of fermentation feedstock.

Another object is to provide an improved method of fermenting carbohydrate to ethanol in a fermentor, comprising:

a) mixing a fermentation feedstock with a fermentation broth containing yeast and/or an enzyme,

b) treating said mixture by adding a composition to the fermentor containing:
10-90 wt. % of an aldehyde selected from the group consisting of formaldehyde, para-formaldehyde, glutaraldehyde, another antimicrobial aldehyde, and mixtures thereof,
1-50 wt. % of a surfactant having an HLB from 4 to 18,
0-20 wt. % of an antimicrobial terpene, or essential oils,
1-50 wt. % of organic acids selected from C₁ to C₂₄ fatty acids, their salts, glycerides and esters thereof, and
1-50 wt. % water;
   wherein the concentration of aldehyde in the fermentor is from about 0.25 to 3 kg/MT of fermentation feedstock, and

c) isolating ethanol,

d) collecting material remaining after fermentation and adding it to animal feed.

Another object of the invention is to provide a method for preventing biofilms formation during the entire process of ethanol production by adding a composition to the liquid slurry or fermentable broth comprising:

a) 10-90 wt.% of an aldehyde selected from the group consisting of formaldehyde, para-formaldehyde, glutaraldehyde, other antimicrobial aldehyde and mixtures thereof,

b) 1-50 wt. % of a surfactant having an HLB from 4 to 18,

c) 1-20 wt. % of an antimicrobial terpene, or essential oils,

d) 1-50 wt % of organic acids selected from C₁ to C₂₄ fatty acids, their salts, glycerides and esters thereof, and

e) 1-50 wt % water,

wherein the concentration of aldehyde in the fermentor is from about 0.25 to 3 kg/MT of fermentation feedstock.

Another object of the invention is to provide a method for disrupting already established biofilms on the entire equipment used for ethanol production by adding a composition to the liquid slurry or fermentable broth comprising:

a) 10-90 wt. % of an aldehyde selected from the group consisting of formaldehyde, para-formaldehyde, glutaraldehyde, other antimicrobial aldehyde and mixtures thereof,

b) 1-50 wt. % of a surfactant having an HLB from 4 to 18,

c) 1-20 wt. % of an antimicrobial terpene, or essential oils,

d) 1 -50 wt % of organic acids selected from C₁ to C₂₄ fatty acids, their salts, glycerides and esters thereof, and

e) 1-50 wt % water,

wherein the concentration of aldehyde in the fermentor is from about 0.25 to 3 kg/MT of fermentation feedstock.

Another object of the invention is to reduce the use of antibiotics and sulfuric acid during the fermentation of carbohydrates adding to the fermentation system a composition comprising:

a) 10-90 wt. % of an aldehyde selected from the group consisting of formaldehyde, para-formaldehyde, glutaraldehyde, other antimicrobial aldehyde and mixtures thereof,

b) 1-50 wt. % of a surfactant having an HLB from 4 to 18

c) 1-20 wt. % of an antimicrobial terpene, or essential oils,

d) 1-50 wt % of organic acids selected from C₁ to C₂₄ fatty acids, their salts, glycerides and esters thereof, and

e) 1-50 wt % water,

wherein the concentration of aldehyde in the fermentor is from about 0.25 to 3 kg/MT of fermentation feedstock.

Another object of the invention is to reduce the antibiotic presence in the resulting sub-product of carbohydrates fermentation e.g. distilled grains, corn gluten and others.

Another object is to reduce antibiotic residues in animal products by feeding the animals sub-products of fermentation resulting from non-antibiotics but the present invention treated substrates.

Another object is to inhibit the development of antibiotic-resistant strains of bacteria which occur during fermentation.

Another object is to increase the yield of ethanol from fermented carbohydrate.

Another object is to improve yeast viability by decreasing the used of sulfuric acid and yeast prewash to decrease bacteria level.

### DESCRIPTION OF THE PREFERRED EMBODIMENTS

### Definitions

“Weight percent” (wt. %) of a component is based on the total weight of the formulation or composition in which the component is included.

“Aldehyde” includes formaldehyde, paraformaldehyde, and other biocidal aldehydes.

“Organic acid” includes formic, acetic, propionic, butyric and other C₁ to C₂₄ fatty acids, or mono-, di-, or triglycerides of C₁ to C₂₄ organic fatty acids or their alkyl esters.

“Antimicrobial terpene” can include allyl disulfide, citral, pinene, nerol, geraniol, carvacrol, eugenol, carvone, anethole, camphor, menthol, limonene, farnesol, carotene, thymol, borneol, myrcene, terpenene, linalool, or mixtures thereof. More specifically, the terpenes may comprise allyl disulfide, thymol, citral, eugenol, limonene, carvacrol, and carvone, or mixtures thereof. The terpene component may include other terpenes with anti-microbial properties and essential oils.

Bacteria that may interfere with ethanol fermentation include Lactobacillus spp. and Leuconostoc spp., which cause the most problems. Other such bacteria include Pediococcus spp., Staphylococcus spp., Streptococcus spp., Bacillus spp. and Clostridia spp. and other bacteria which reduce fermentation efficiency.

In ethanol produced from corn, antibiotics are the common biocide, e.g., virginiamycin, penicillin, clindamycin, tylosin, chloramphenicol, cephalosporin and tetracycline. However, because the end product is not fed to animals when ethanol is produced from sugarcane, other biocides can be used since residues do not present the same problem. In such cases suitable biocides include carbamates, quaternary ammonium compounds, phenols and antibiotics (e.g., virginiamycin, penicillin, clindamycin, tylosin, chloramphenicol, cephalosporin and tetracycline).

The term “effective amount” of a compound means an amount capable of performing the function or having the property for which the effective amount is expressed, such as a non-toxic but sufficient amount to provide anti-microbial benefits in a biofilm preventer or disrupter. Thus an effective amount may be determined by one of ordinary skill in the art by routine experimentation.

Formulations vary not only in the concentrations of the major components, e.g., aldehydes and organic acids, but also in the type of terpenes, surfactant(s) and water concentration. This invention can be modified by adding or deleting the terpene, type of organic acid, and using other types of surfactant.

### Composition(s)

In general, a composition of the invention contains:

a) 10-90 wt.% of an aldehyde selected from the group consisting of formaldehyde, para-formaldehyde, glutaraldehyde, other antimicrobial aldehyde and mixtures thereof,

b) 1-50 wt. % of a surfactant having an H LB from 4 to 18,

c) 1-20 wt. % of an antimicrobial terpene, or essential oils,

d) 1-50 wt. % of an organic acid or mixtures of organic acids selected from acetic, propionic, butyric, or other C₁ to C₂₄ fatty acids, salt forms, glycerides and esters thereof, and,

e) 1-50 wt % water.

The antimicrobial terpenes, plant extracts or essential oils containing terpenes can be used in the compositions of this invention as well as the more purified terpenes. Terpenes are readily available commercially or can be produced by methods known in the art, such as solvent extraction or steam extraction/distillation or chemical synthesis.

The surfactant is non-ionic including ethoxylated castor oil surfactants with 1 to 200 ethylene molecules distributed normally around the mean, preferably a mean of 10 to 80. Other surfactants with similar characteristics can be used including polysorbates surfactants.

### Methods

The present invention is effective against bacteria and bacterial biofilms. Examples of such infective agents include, E. coli, Salmonella spp., Clostridium spp., Campylobacter spp., Shigella spp., Brachyspira spp., Listeria spp., Arcobacter spp, Lactobacillus, Pediococcus, Staphylococcus, Enterococcus, Acetobacter, Gluconobacter, A. pasterurianus, B. Subtilis, Leuconostoc mesenteroides, Weissella paramesenteroides and others.

The mixture of the present invention is applied by a spray nozzle.

The mixture of the present invention is applied mixed with a soluble carrier to the fermentable carbohydrate.

The mixture of the present invention is applied mixed in a starch-based carrier to the fermentable carbohydrate.

The mixture of the present invention is mixed with a liquid or solid carrier prior to be added to the fermentable carbohydrate.

The mixture of the present invention is applied drop-wise on the fermentable broth or slurry.

The mixture of the present invention is applied by inline injection to the fermentable broth or slurry.

The mixture of the present invention is applied in any or all of the treatable areas during production of sugar and ethanol from sugarcane.

The mixture of the present invention is applied in any or all of the treatable areas during production of sugar and ethanol from sugar beet.

The mixture of the present invention is applied in any or all of the treatable areas during production of sugar and ethanol from corn, other starchy or cellulosic material.

The mixture is applied so as to provide a uniform and homogeneous distribution throughout the carbohydrate substrate.

Various patents and publications are referenced throughout this specification. The disclosures of each document are hereby incorporated by reference in their entirety.

### EXAMPLE 1

This example shows the base formulation “A” product used in subsequent examples

TABLE 1
Components of Formulation “A”

| Ingredient | (%) |
|---|---|
| Formalin (37%) | 90.00 |
| Propionic Acid | 9.00 |
| d-limonene (terpene) | 0.40 |
| Polysorbate 80 (surfactant) | 0.60 |

### EXAMPLE 2

The objective of this study was to determine the effect of a Formula “A” on the survival of Lactobacillus. Lactobacillus plantarum (B-4496) obtained from USDA-Microbial Genomics and Bioprocessing Research in Illinois was grown in Difco™ Lactobacilli spp. MRS (Man-Rogosa-Sharpe) broth. The broth culture was diluted with sterile peptone water to obtain different concentrations of Lactobacillus. Dilutions were treated with different concentrations of Formula A (0, 1, 2 and 3 kg/MT) and incubated for 24 hours at room temperature (20° C.). After incubation, triplicate samples were taken and plated on MRS broth containing 1.5% Difco™ Agar Granulated solidifying agent. Plates were incubated at 37° C. for 24 hours before enumeration of colonies. The average cfu/ml for each treatment is shown in Table 2.

TABLE 2
Effect of Formula “A” in the Growth of Lactobacillus
(cfu/ml)

| | | | | | | | |
|---|---|---|---|---|---|---|---|
| Control (0 kg/MT) | 4.1 × 10⁷ | 4.8 × 10⁶ | 5.2 × 10⁵ | 4.8 × 10⁴ | 3.3 × 10² | 5.3 × 10¹ | 4.0 |
| 1 kg/MT | 5.0 × 10⁷ | 1.2 × 10⁶ | 8.6 × 10⁵ | 7.9 × 10³ | 0 | 0 | 0 |
| 2 kg/MT | 0 | 0 | 0 | 0 | 0 | 0 | 0 |
| 3 kg/MT | 0 | 0 | 0 | 0 | 0 | 0 | 0 |

It was observed that the use 2 kg/MT of the Formula “A” eliminated the growth of Lactobacillus in a culture containing 10⁷ cfu/ml.

### EXAMPLE 3

The objective of this study was to determine the effect of Formula “A” on the survival of yeast and Lactobacillus during fermentation. Sterile, finely ground corn was mixed with sterile water in a glass fermenter. Next, a commercial enzyme solution containing alpha-amylase and glucoamylase blend (Stargen: Genencor) for processing of uncooked starch was added. Fali Yeast (10¹⁰ cfu/g; Fleischmann) used as fermentative yeast was added to the corn slurry mixtures while mixing. Finally, Lactobacillus plantarum (B-4496), obtained from USDA-Microbial Genomics and Bioprocessing Research in Illinois and grown in Difco™ Lactobacilli spp. MRS broth, was used as the representative bacterial contaminant of the fermenter. Formula “A” at a dose of 1 Kg/MT was added as the final step before starting the fermentation process. Samples of the liquid phase taken at 4 h, 24 h, 48 h, 72 h and 96 hours were analyzed for yeast and lactobacillus counts. The results are shown in the following tables:

TABLE 3
Effect of Formula “A” on Yeast
Counts During Fermentation (cfu/ml)

| | 4 h | 24 h | 48 h | 72 h | 96 h |
|---|---|---|---|---|---|
| Control | 6.8 × 10⁸ | 1.8 × 10⁹ | 2.3 × 10⁸ | 8.0 × 10⁸ | 8.0 × 10¹¹ |
| Formula A | 7.9 × 10⁸ | 2.3 × 10⁹ | 4.8 × 10⁸ | 8.0 × 10⁸ | 2.0 × 10⁹ |
| (1 kg/MT) | | | | | |

TABLE 4
Effect of Formula “A” on Lactobacillus
Counts During Fermentation (cfu/ml)

| | 4 h | 24 h | 48 h | 72 h | 96 h |
|---|---|---|---|---|---|
| Control | 7.6 × 10⁵ | 1.6 × 10⁸ | 1.3 × 10⁹ | 2.9 × 10¹² | 2.2 × 10⁸ |
| Formula A | 6.4 × 10⁵ | 6.8 × 10⁷ | 1.6 × 10⁹ | 1.6 × 10¹² | 9.0 × 10⁷ |
| (1 kg/MT) | | | | | |

It was observed that 1 kg/ton of the formaldehyde-based product decreased the level of Lactobacillus, but did not affect the level of yeast.

### EXAMPLE 4

The objective of this study was to determine if changes in Formula “A” resulted in similar benefits as previous examples. Fermentation solution was free of Lactobacillus. Formula “A” was modified as described in Table 5. This example was also conducted as to simulate sugar cane fermentation.

TABLE 5
Changes compared to Formula “A”

| | A | B | C | D | E |
|---|---|---|---|---|---|
| Formaldehyde (37%) | 90 | 90 | 90 | 90 | 90 |
| Propionic acid | 9 | 9 | 8 | 5 | 0 |
| d-limonene | 0.4 | 0 | 0 | 0 | 0 |
| Polysorbate 80 | 0.6 | 1 | 2 | 5 | 10 |

In 250-ml glass fermentors, 100 ml of a 12% sterile sucrose solution, 10 ml yeast (10⁶ cfu/ml) and 25 ul of each formulation were added and incubated for 24 hours. After incubation, samples were taken for the determination of ethanol yield. The results are shown on Table 6.

TABLE 6
Effect of Different Formulations on Ethanol Yield (% Ethanol)

| | |
|---|---|
| Control | 5.97 ± 0.10^{x} |
| Formula “A” | 5.59 ± 0.00^{y} |
| Formula “B” | 5.66 ± 0.06^{xy} |
| Formula “C” | 5.84 ± 0.30^{xy} |
| Formula “D” | 5.80 ± 0.06^{xy} |
| Formula “E” | 5.94 ± 0.00^{xy} |

When there is no bacterial competition during fermentation, the concentration of ethanol was similar in all treatments with the exception of Formula A.

### EXAMPLE 5

The objective of this study was to determine if changes in Formula “A” resulted in similar benefits as shown on previous examples. In this example, Lactobacillus was added to the fermentors to simulate naturally occurring Lactobacillus. The same formulations as Example 4 were used. In 250-ml glass fermentors, 100 ml of a 12% sterile sucrose solution, 10 ml yeast (10⁶ cfu/ml) and 25 ul of each formulation were added and incubated for 24 hours. After incubation samples were taken for the determination of ethanol yield as well as yeast and lactobacillus. The results are shown on table 7.

TABLE 7
Effect of Effect of Different Formulations
on Ethanol Yield and Microbial Profile

| | | | % ethanol |
|---|---|---|---|
| Formulations | Yeast | Lactobacillus | (mean ± S.D.) |
| Control | 1.45 × 10⁸ | 1.20 × 10⁸ | 6.18 ± 0.38^{xy} |
| Formula “A” | 1.08 × 10⁸ | 1.13 × 10⁸ | 6.38 ± 0.15^{xy} |
| Formula “B” | 9.78 × 10⁷ | 1.16 × 10⁸ | 6.28 ± 0.50^{xy} |
| Formula “C” | 7.30 × 10⁷ | 1.01 × 10⁸ | 6.67 ± 0.20^{x} |
| Formula “D” | 8.12 × 10⁷ | 7.77 × 10⁷ | 5.06 ± 0.02^{y} |
| Formula “E” | 8.20 × 10⁷ | 9.97 × 10⁷ | 5.49 ± 0.37^{xy} |

It was observed that formulas A, B and C resulted in a numerical improvement in ethanol yield in the presence of bacterial completion when fermentation lasted 24 hours.

### EXAMPLE 6

The objective of this study was to determine if changes in Formula “A” resulted in similar benefits as shown in previous examples. In this example, Lactobacillus was added to the fermentors to simulate naturally occurring Lactobacillus. The same formulations as Example 4 were used. In 250-ml glass fermentors, 100 ml of a 12% sterile sucrose solution, 10 ml yeast (10⁶ cfu/ml) and 25 ul of each formulation were added and then incubated for 18 hours. After incubation, samples were taken for the determination of ethanol yield as well as yeast and lactobacillus. The results are shown in Table 8.

TABLE 8
Effect of Effect of Different Formulations
on Ethanol Yield and Microbial Profile

| | | % ethanol |
|---|---|---|
| Formulations | Yeast | (mean ± S.D.) |
| Control | 1.27 × 10⁸ | 5.41 ± 0.16^{xy} |
| Formula “A” | 1.11 × 10⁸ | 5.11 ± 0.12^{y} |
| Formula “B” | 1.08 × 10⁸ | 5.14 ± 0.08^{y} |
| Formula “C” | 1.23 × 10⁸ | 5.37 ± 0.15^{xy} |
| Formula “D” | 1.27 × 10⁸ | 5.56 ± 0.31^{x} |
| Formula “E” | 1.34 × 10⁸ | 5.22 ± 0.07^{y} |

It was observed that formula D resulted in an improvement in ethanol yield in the presence of bacterial completion when fermentation lasted 18 hours.

### EXAMPLE 7

The objective of this example was to determine the effect of the using Formula “A” on the destruction of biofilms using lactobacillus as the biofilm forming bacteria. Formula “A” was added at a dose of 0.5 or 1 Kg/MT. The formation of biofilms was prepared as follows:

In 96-well polystyrene plates: 100 μl of Lactobacillus culture in nutrient broth was added to each well and incubated for 48 hours at 37° C. in an anaerobic chamber. After incubation the plates were washed 5 times with distilled water and blotted dry. After drying, 100 ul of formulation “A” was added to the wells, incubated for 4 or 24 hours at 37° C. in an anaerobic chamber and then washed 5 times with distilled water. After blotting dry, 30 μl of 1% crystal violet was added then incubated for 15 minutes at room temperature to allow the dyeing of biofilms. Wells were washed 5 times with distilled water, blotted dry, 200 μl of 95% ethanol was added and then the plates were read at 590 nm. Results are expressed as the % difference between O.D. of control and the treated samples.

TABLE 9
Biofilms Destruction when Exposed for 4 hours

| | O.D. | % Destruction |
|---|---|---|
| Control | 0.746 | — |
| 0.5 Kg/MT | 0.547 | 27 |

TABLE 10
Biofilms Destruction when Exposed for 4 hours

| | O.D. | % Destruction |
|---|---|---|
| Control | 0.803 | — |
| 1.0 Kg/MT | 0.691 | 14 |

TABLE 11
Biofilms Destruction when Exposed for 24 hours

| | O.D. | % Destruction |
|---|---|---|
| Control | 0.396 | — |
| 0.5 Kg/MT | 0.344 | 13 |
| 1.0 Kg/MT | 0.312 | 20 |

Both dosifications of Formula “A” resulted in a partial destruction of established biofilms.

### EXAMPLE 8

The objective of this example was to determine the effect of the formulas from Example 4 on the destruction of biofilms using Lactobacillus as the biofilm forming bacteria. All formulas were added at a dose of 1 Kg/MT. The formation of biofilms was prepared as follows:

In 96-well polystyrene plate: 100 μl of Lactobacillus culture in nutrient broth was added to each well and incubated for 48 hours at 37° C. in an anaerobic chamber. After incubation the plates were washed 5 times with distilled water and blotted dry. After drying, 100 ul of each formulation were added to the wells, the plates incubated for 4 hours at 37° C. in an anaerobic chamber and then washed 5 times with distilled water. After blotting dry, 30 μl of 1% crystal violet was added and the plates incubated for 15 minutes at room temperature to allow the dyeing of the biofilm. Wells were washed 5 times with distilled water, blotted dry, 200 μl 95% ethanol was added and then the plates were read at 590 nm. Results are expressed as the % difference between O.D. of control and the treated samples.

TABLE 12
Biofilms Destruction when Exposed for 4 hours

| | O.D. | % Destruction |
|---|---|---|
| Control | 0.076 | — |
| Formula “A” | 0.052 | 32 |
| Formula “B” | 0.055 | 28 |
| Formula “C” | 0.055 | 28 |
| Formula “D” | 0.051 | 33 |
| Formula “E” | 0.051 | 33 |

All formulations were effective against established biofilms.

### EXAMPLE 9

The objective of this example was to determine the effect of the formula “A” cited in the previous examples on the prevention of biofilms formation using Lactobacillus as the biofilm forming bacteria. Formula “A” was added at a dose of 0.5 and 1 Kg/MT. The prevention of biofilms formation was prepared as follows:

In 96-well polystyrene plate: 100 μl of Lactobacillus culture in nutrient broth and 100 ul of each formula “A” at a dose of 0.5 or 1.0 Kg/MT were added to the wells and incubated for 48 hours at 37° C. in an anaerobic chamber. After incubation the plates were washed 5 times with distilled water and blotted dry. After blotting dry, 30 μl of 1% crystal violet was added, then the plates were incubated for 15 minutes at room temperature to allow the dyeing of biofilms. Wells were washed 5 times with distilled water, blotted dry, 200 μl 95% ethanol was added and then the plates were read at 590 nm. Results are expressed as the % difference between O.D. of control and the treated samples.

TABLE 13
Biofilms Prevention when Exposed for 48 hours

| | O.D. | % Prevention |
|---|---|---|
| Control | 0.775 | — |
| 0.5 Kg/MT | 0.674 | 13 |
| 1.0 Kg/MT | 0.264 | 66 |

Formula “A” at both doses reduced the establishment of biofilms, with 1 Kg/MT being more effective than 0.5 Kg/MT.

### EXAMPLE 10

The objective of this example was to determine the effect of the formulas from Example 4 on the prevention of biofilms formation using Lactobacillus as the biofilm forming bacteria. All formulas were added at a dose of 1 Kg/MT. The prevention of biofilms formation was prepared as follows:

In 96-well polystyrene plate: 100 μl of Lactobacillus culture in nutrient broth and 100 ul of each formula at a dose of 1.0 Kg/MT were added to the wells and incubated for 36 hours at 37° C. in an anaerobic chamber. After incubation the plates were washed 5 times with distilled water and blotted dry. After blotting dry, 30 μl of 1% crystal violet was added then incubated for 15 minutes at room temperature to allow the dyeing of biofilms. Wells were washed 5 times with distilled water, blotted dry, 200 μl 95% ethanol was added and then the plates were read at 590 nm. Results are expressed as the % difference between O.D. of control and the treated samples.

TABLE 14
Biofilms Prevention when Exposed for 48 hours

| | O.D. | % Prevention |
|---|---|---|
| Control | 0.113 | — |
| Formula “A” | 0.094 | 17 |
| Formula “B” | 0.093 | 18 |
| Formula “C” | 0.073 | 35 |
| Formula “D” | 0.077 | 32 |
| Formula “E” | 0.079 | 30 |

All formulas decreased the establishment of biofilms.

### EXAMPLE 11

The objective of this example was to determine ethanol production using Formula “A” treated corn or Formula “A” added into the fermenters.

Whole corn was treated with zero (control) or 0.50 kg/MT, and stored overnight before grinding and setting the fermentation procedure. Treated and un-treated ground corn were mixed with water and incubated at room temperature in an anaerobic environment for 6 hours. Formulation A was added to the fermenters before the 6 hour incubation. The other reagents were added in the fermenters as described in the following.

| | Corn | Water | Enzyme | Yeast |
|---|---|---|---|---|
| Treatment | (gr) | (ml) | (ml) | (10⁸ cfu/gr) |
| Control - 0 kg/MT | 30 | 100 | 0.20 | 1.0 gr |
| Formulation A - 0.50 kg/MT | 30 | 100 | 0.20 | 1.0 gr |
| Formulation A - 30 | 30 | 100 | 0.20 | 1.0 gr |
| ul at fermenter, | | | | |
| (0.50/MT of corn) | | | | |

Yeast was hydrated with lukewarm water at 1 gr/10 ml prior to adding to fermenters. Fermenters were kept under constant stirring (low speed) at room temperature for 72 hours before sampling for yeast and alcohol production. After 72 hours, triplicate samples/fermenter were taken and plated on PDA for the determination of yeast count. Plates were incubated at 27° C. for 48 hours and colonies enumerated.

### Results:

| | Yeast | % |
|---|---|---|
| Treatment | (cfu/gr) | ethanol |
| Control | 8.69 × 10⁸ | 9.95 ± 0.13 |
| Formula “A” treated corn | 8.13 × 10⁸ | 10.60 ± 0.89 |
| Formula “A” treatment at | 7.94 × 10⁸ | 12.05 ± 0.16 |
| fermentors | | |

The addition of Formulation A in the fermenters improved ethanol yield as compared to Formula “A” treated corn.

It will be apparent to those skilled in the art that variations and modifications of the invention can be made without departing from the spirit and scope of the teachings above.

It is intended that the specification and examples be considered as exemplary only and are not restrictive.

1. A high yield method of fermenting carbohydrate to ethanol in a fermentor, comprising:
a) mixing a fermentation feedstock with a fermentation broth containing yeast and/or an enzyme,
b) treating said mixture by adding a composition to the fermentor containing:
10-90 wt. % of an aldehyde selected from the group consisting of formaldehyde, para-formaldehyde, glutaraldehyde, another antimicrobial aldehyde, and mixtures thereof,
1-50 wt. % of a surfactant having an HLB from 4 to 18,
0-20 wt. % of an antimicrobial terpene, or essential oils,
1-50 wt. % of organic acids selected from C₁ to C₂₄ fatty acids, their salts, glycerides and esters thereof, and
1-50 wt. % water;
wherein the concentration of aldehyde in the fermentor is from about 0.25 to 3 kg/MT of fermentation feedstock, and
c) isolating ethanol.

2. The method of claim 1, wherein the fermentation feedstock is corn, sorghum, wheat, triticale, rye, barley, rice or tubers.

3. The method of claim 1, wherein the fermentation feedstock is sugar cane or sugar beet.

4. The method of claim 1, wherein the carbohydrate to be fermented is derived from cellulose.

5. The method claim 1, wherein development of antibiotic-resistant strains of bacteria is inhibited.

6. A fermentation broth or slurry, comprising:
a) carbohydrate feedstock to be fermented, yeast, and/or an enzyme, and
b) a treatment composition containing:
10-90 wt. % of an aldehyde selected from the group consisting of formaldehyde, para-formaldehyde, glutaraldehyde, another antimicrobial aldehyde and mixtures thereof,
1-50 wt. % of a surfactant having an HLB from 4 to 18,
1-20 wt. % of an antimicrobial terpene, or essential oils,
1-50 wt. % of organic acids selected from C₁ to C₂₄ fatty acids, their salts, glycerides and esters thereof, and
1-50 wt. % water;
wherein the concentration of aldehyde is from about 0.25 to 3 kg/MT of fermentation feedstock.

7. The fermentation broth of claim 6, wherein the carbohydrate feedstock is corn, sorghum, wheat, triticale, rye, barley, rice or tubers, and the aldehyde is formaldehyde with a concentration of 0.25 to 3.0 kg/MT.

8. The fermentation broth of claim 6, wherein the carbohydrate feedstock is sugar cane or sugar beet.

9. The fermentation broth of claim 6, wherein the carbohydrate feedstock is derived from cellulose.

10. The fermentation broth of claim 6, wherein development of antibiotic-resistant strains of bacteria is inhibited.

11. An improved method of fermenting carbohydrate to ethanol in a fermentor, comprising:
a) mixing a fermentation feedstock with a fermentation broth containing yeast and/or an enzyme,
b) treating said mixture by adding a composition to the fermentor containing:
10-90 wt. % of an aldehyde selected from the group consisting of formaldehyde, para-formaldehyde, glutaraldehyde, another antimicrobial aldehyde, and mixtures thereof,
1-50 wt. % of a surfactant having an HLB from 4 to 18,
0-20 wt. % of an antimicrobial terpene, or essential oils,
1-50 wt. % of organic acids selected from C₁ to C₂₄ fatty acids, their salts, glycerides and esters thereof, and
1-50 wt. % water;
wherein the concentration of aldehyde in the fermentor is from about 0.25 to 3 kg/MT of fermentation feedstock, and
c) isolating ethanol,
d) collecting material remaining after fermentation and adding it to animal feed.

12. The method of claim 11, wherein the organic acid is formic, acetic, propionic, or butyric.

13. The method of claim 11, comprising an antibiotic to control bacteria in an amount less than its MIC in fermentations without composition b).

14. The method of claim 11, which is free of antibiotic used to control bacteria in fermentation.

15. The method of claim 11, wherein bacteria comprise Lactobacillus spp. E. coli, Salmonella spp., Clostridium spp., Campylobacter spp., Shigella spp., Brachyspira spp., Listeria spp., Arcobacter spp, Pediococcus, Staphylococcus, Enterococcus, Acetobacter, Gluconobacter, A. pasterurianus, B. Subtilis, Leuconostoc mesenteroides, Weissella paramesenteroides and bacteria able to produce biofilms in solid surfaces.

16. The method of claim 11, which is free of virginiamycin or sulfuric acid.

17. The method of claim 11, wherein the carbohydrate feedstock is corn, sorghum, wheat, triticale, rye, barley, rice or tubers, and the aldehyde is formaldehyde with a concentration of 0.25 to 3.0 kg/MT.

18. The method of claim 11, wherein the carbohydrate feedstock is sugar cane or sugar beet.

19. The method of claim 11, wherein the carbohydrate feedstock is derived from cellulose.

20. The method of claim 11, wherein development of antibiotic-resistant strains of bacteria is inhibited.
